(19) United States
(12) Patent Application Publication
(10) Pub. No.: US 2017/0225170 A1
(43) Pub. Date: Aug. 10, 2017

(54) IMMUNOBLOTTING TEST BOARD REACTION DEVICE

(71) Applicant: Hangzhou Zheda Dixun Biological Gene Engineering Co., LTD., Zhejiang (CN)

(72) Inventors: Shandong Wu, Zhejiang (CN); Huahao Shen, Zhejiang (CN); Yi Liu, Zhejiang (CN); Zhoujie Wu, Zhejiang (CN); Mingzhi Zhu, Zhejiang (CN)

(21) Appl. No.: 15/303,891

(22) PCT Filed: Apr. 1, 2015
(86) PCT No.: PCT/CN2015/075666

§ 371 Date: Oct. 13, 2016

(30) Foreign Application Priority Data
Oct. 14, 2014 (CN) 201420592921.7

(51) Int. Cl. B01L 9/00; G01N 35/00; G01N 33/543

(52) U.S. Cl. CPC B01L 9/52; B01L 9/50; G01N 33/54366; G01N 35/00029; B01L 2200/025; B01L 2300/0609; B01L 2300/0809; B01L 2200/141

(57) ABSTRACT

An immunoblotting test board reaction device includes a tray rack and at least one tray, wherein a plurality of grooves for receiving test boards are provided in the upper surface of each tray and tray back bayonets (24) for placing the tray onto a full-automatic immunoblotting instrument are provided on the bottom surface of the tray. The tray rack includes tray grooves and snap bayonets so that the trays and the test boards can be installed and removed conveniently, and the test boards can be always in a steady state no matter in a washing process or an incubating process. Moreover, a plurality of samples in their corresponding test boards can be scanned at a time, thereby facilitating the processing of test results.

### CROSS-REFERENCE TO RELATED APPLICATIONS

This application in a national phase of PCT/CN2015/075666, filed on Apr. 1, 2015, which claims priority to Chinese Application No. 201420592921.7, which was filed Oct. 14, 2014. These prior applications are incorporated herein by reference, in their entireties.

### TECHNICAL FIELD

The present invention relates to an immunoblotting test board reaction device.

### BACKGROUND

At present, immunoblotting products tend to be detected by full-automatic operation instruments, which are highly safe and simple to operate. However, these instruments, with relatively high costs, are not suitable for use in general basic medical institutions. Both of EUROBlotMasterII produced by German EUROIMMUN MEDIZINISCHE LABORDIAGNOSTIKAAG and Blot Processor produced by Tecan Austria GmbH are equipped with immunoblotting membrane strip reaction trays, which are only suitable for testing bare strips but not suitable for test boards with reaction grooves; in addition, the process of sticking the blot bare strips takes much time and energy, and there exists biohazard risk when an operator's hand comes into contact with strips. The immunoblotting test board reaction bracket invented by Mediwiss Analytic GmbH can well fix test boards to implement the steps of washing, replacing liquid and the like in manual operation. The reaction bracket can be used for simultaneously testing a plurality of samples, so the application range is wide. However, the reaction bracket achieves installation and removal of test boards by means of insertion and pull-out actions, and the insertion and pull-out processes are relatively laborious due to small stressed area. Moreover, fingers are apt to touch the surfaces of membrane in reaction grooves during insertion and pull-out to cause background pollution, and clamping grooves are apt to be broken after multiple times of insertion and pull-out.

### SUMMARY

In order to solve the problems that the present full-automatic operation instruments are high in cost, the existing reaction brackets are laborious to be inserted and pulled out, fingers are apt to touch the surfaces of membrane in reaction grooves during insertion and pull-out to cause background pollution, and clamping grooves are apt to be broken after multiple times of insertion and pull-out, the present invention provides an immunoblotting test board reaction device, which can facilitate installation and removal of test boards, avoid laborious insertion and pull-out of the test boards, meet manual and instrument operation simultaneously, and has strong practicability and wide application range.

An immunoblotting test board reaction device of the present invention comprises a tray rack and at least one tray, wherein, a plurality of grooves for receiving test boards are provided in the upper surface of the tray, the floor of each groove equipped with a bayonet for locking the corresponding test board and the groove of the tray fitting snugly to the corresponding test board; a snap button for snapping onto the tray rack is provided on one side of the tray, wherein, the snap button comprises a buckling part and a pressing part, one end of the buckling part is fixed to the edge of the tray, and the other end is fixedly connected to the pressing part; tray back bayonets for placing the tray onto a full-automatic immunoblotting instrument are provided on the bottom surface of the tray; and the tray rack comprises tray grooves and snap bayonets which are arranged at the edges of the tray grooves and can be snap-fitted to the buckling parts of the trays.

In one embodiment, all the grooves of the tray are uniformly arranged on the upper surface of the tray in the same direction, and each groove corresponds to a numbered groove.

In one embodiment, the numbered grooves 25 are solid or hollow, and may be rectangular, square, semicircular, elliptical, fan-shaped, trapezoidal, rhombic, triangular or in other appropriate shape, and may be numbered in the form of Arabic numerals, lower-case Chinese numerals, Roman numerals or English letters, or in other appropriate form.

In one embodiment, the groove of the tray is solid or hollow, and, at its any position, the groove 21 may have at least one square, rectangular, elliptical, circular, rhombic, triangular or trapezoidal hole or at least one hole in other appropriate shape. The quantity of the holes may be 1, 2, 3 or other appropriate number.

In one embodiment, the bayonets may be rectangular, square, semicircle, elliptical, fan-shaped, trapezoidal, rhombic, triangular or in other appropriate shape.

In one embodiment, the buckling part is of groove-type structure which may be U-shaped, V-shaped or in other appropriate shape. The pressing part may be rectangular, square, circular, semicircular, elliptical, fan-shaped, trapezoidal, rhombic, triangular or in other appropriate shape, and the snap bayonet may be matched with the pressing part in shape.

In one embodiment, at its any position, the bottom surface of the tray may be provided with at least one tray back bayonet for support. The tray back bayonet may be circular, rectangular, square, semicircular, elliptical, fan-shaped, trapezoidal, rhombic, triangular or in other appropriate shape.

In one embodiment, each tray groove of the tray rack is solid or hollow. At its any position, the tray groove of the tray rack may have at least one square, rectangular, elliptical, circular, rhombic, triangular or trapezoidal hole or at least one hole in other appropriate shape.

In one embodiment, the trays and the tray rack may be made of ABS, PC, PA, POM, PPE, PETP, PBTP, PPS or other appropriate engineering plastics, or the above described plastics into which 20-30% of or other appropriate ratio of glass fiber material is added.

The present invention has the following beneficial effects: the test boards can be easily installed to or removed from the trays, and the grooves of the tray fits snugly to the test boards, thereby guaranteeing that the test boards are always in a steady state no matter in a washing process or an incubating process. Meanwhile, the reaction trays can be used for both manual and instrument operation. In addition, after completion of an experiment, a plurality of trays can be placed on the tray rack at the same time, such that a plurality of samples in their corresponding test boards can be scanned at a time, thereby greatly facilitating the processing of test results.

### BRIEF DESCRIPTION OF THE DRAWINGS

FIG. 1 is a front view of an embodiment of the present invention;

FIG. 2 is a sectional view on line A-A of FIG. 1;

FIG. 3 is an exploded view of FIG. 1;

FIG. 4 is a sectional view on line B-B of FIG. 1;

FIG. 5 is a first position schematic diagram of a tray, a tray rack and test boards of the present invention;

FIG. 6 is a second position schematic diagram of a tray, a tray rack and test boards of the present invention;

FIG. 7 is a front view of trays installed with a tray rack of the present invention;

FIG. 8 is a sectional view on line A-A of FIG. 7;

FIG. 9 is a sectional view on line B-B of FIG. 7;

FIG. 10 is a front view of the tray rack of the present invention;

FIG. 11 is a bottom view of the tray rack of the present invention;

FIG. 12 is a front view of the tray of the present invention;

FIG. 13 is a bottom view of the tray of the present invention;

FIG. 14 is a first structural diagram of the test board of the present invention;

FIG. 15 is a second structural diagram of the test board of the present invention.

### DETAILED DESCRIPTION

Preferred embodiments of the present invention will be described below in detail with reference to the accompanying drawings.

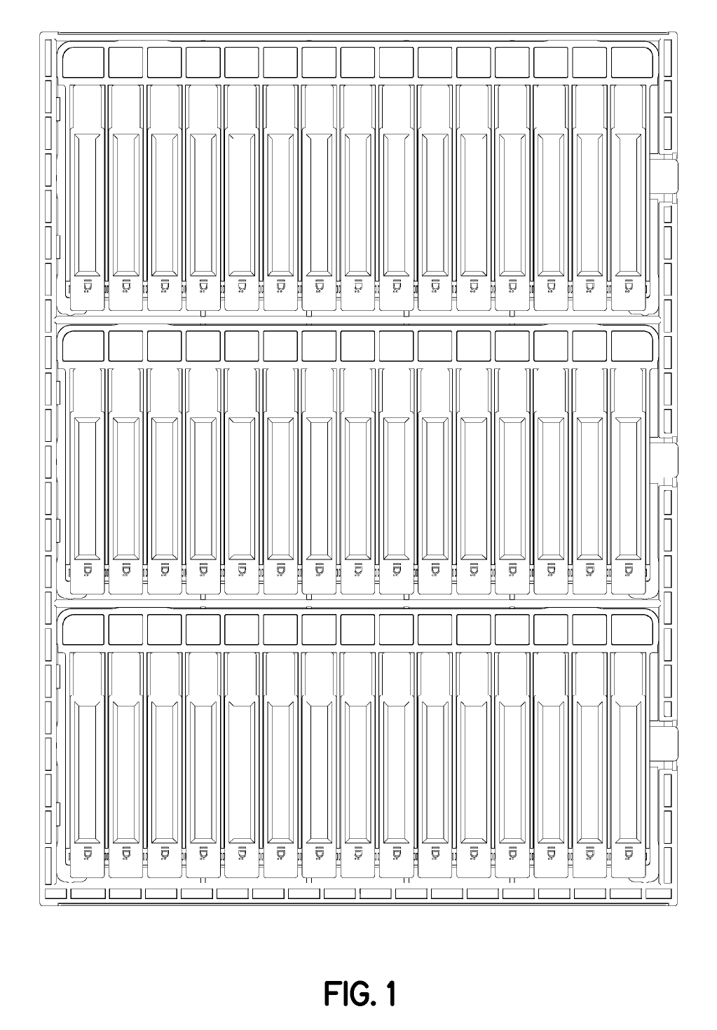

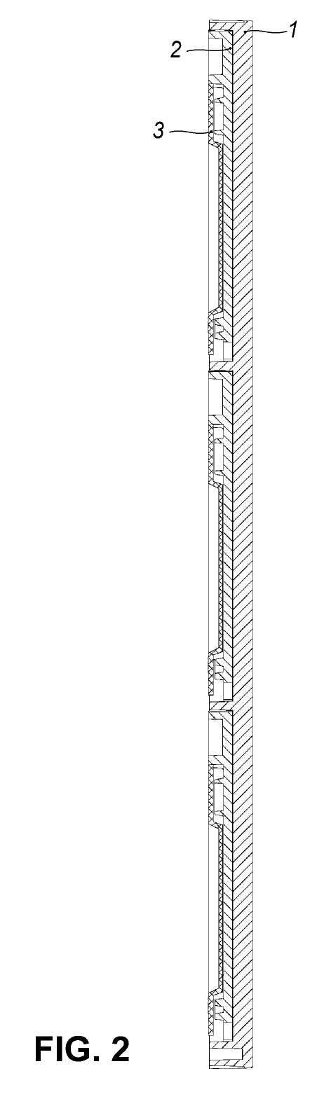

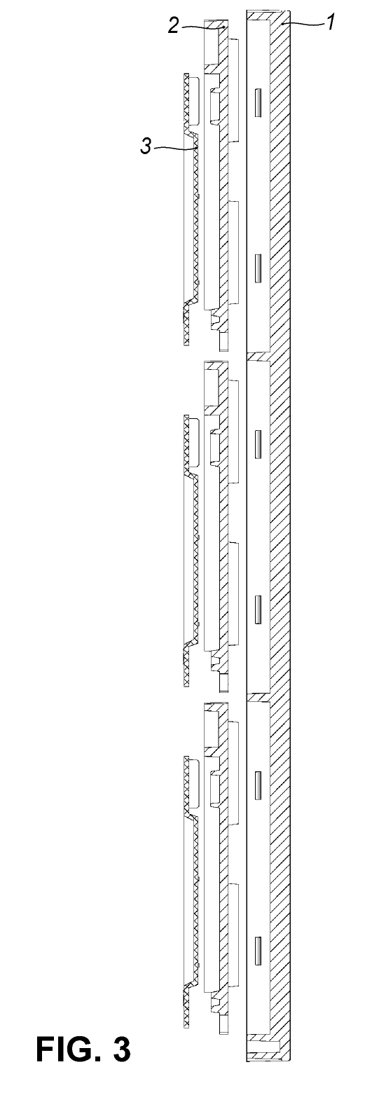

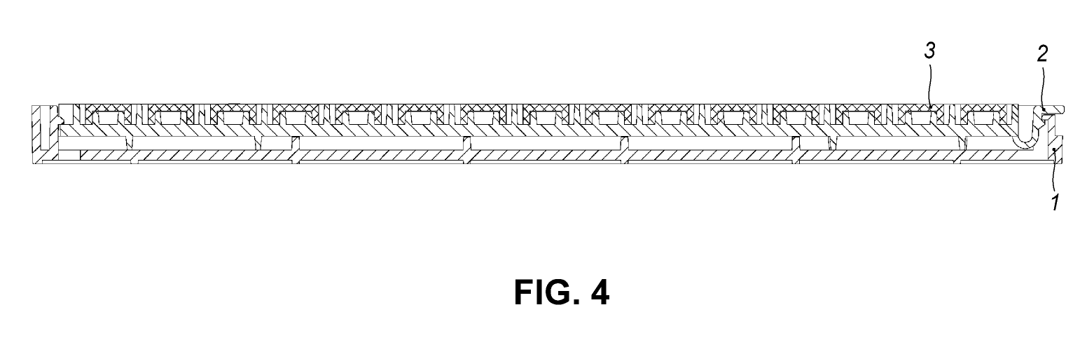

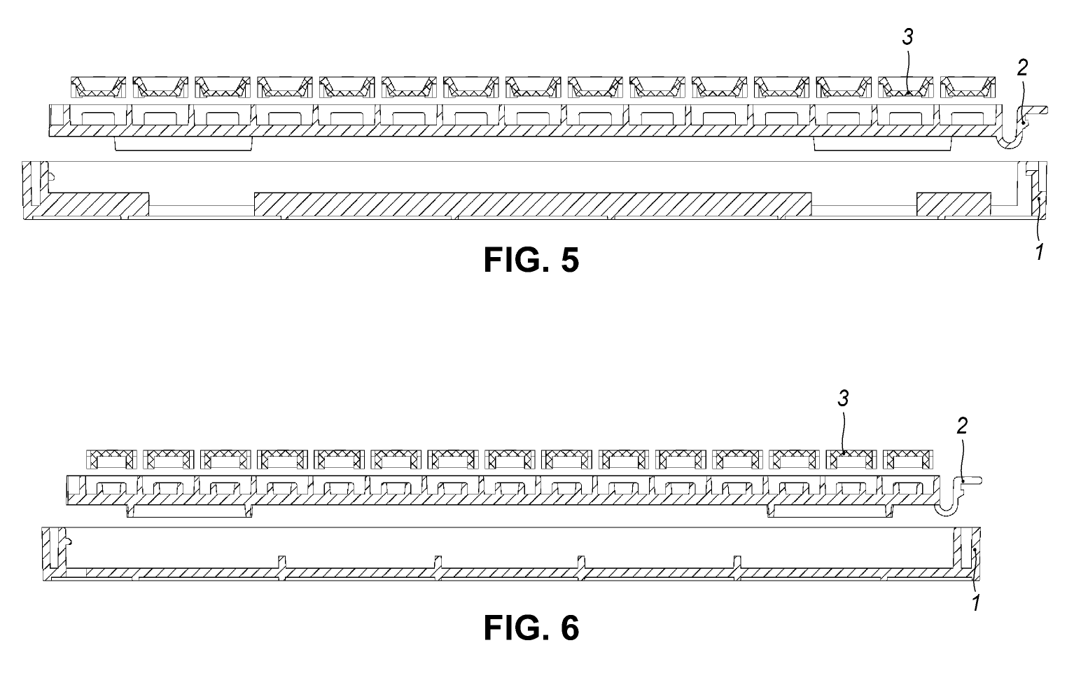

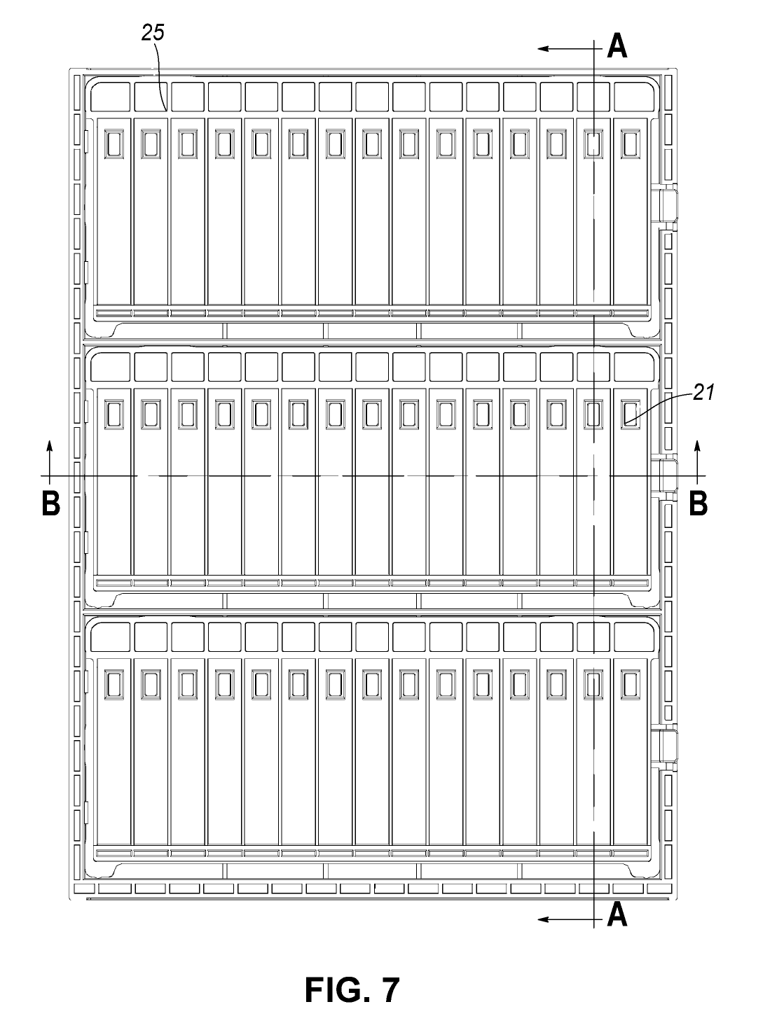

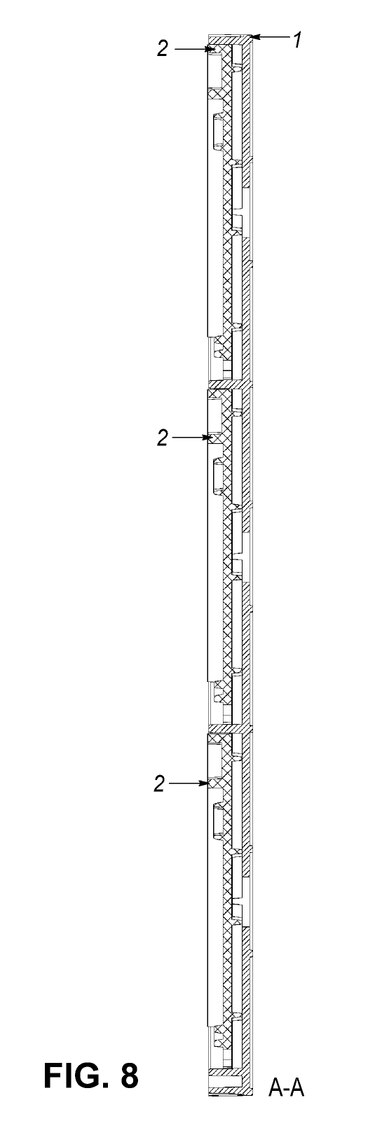

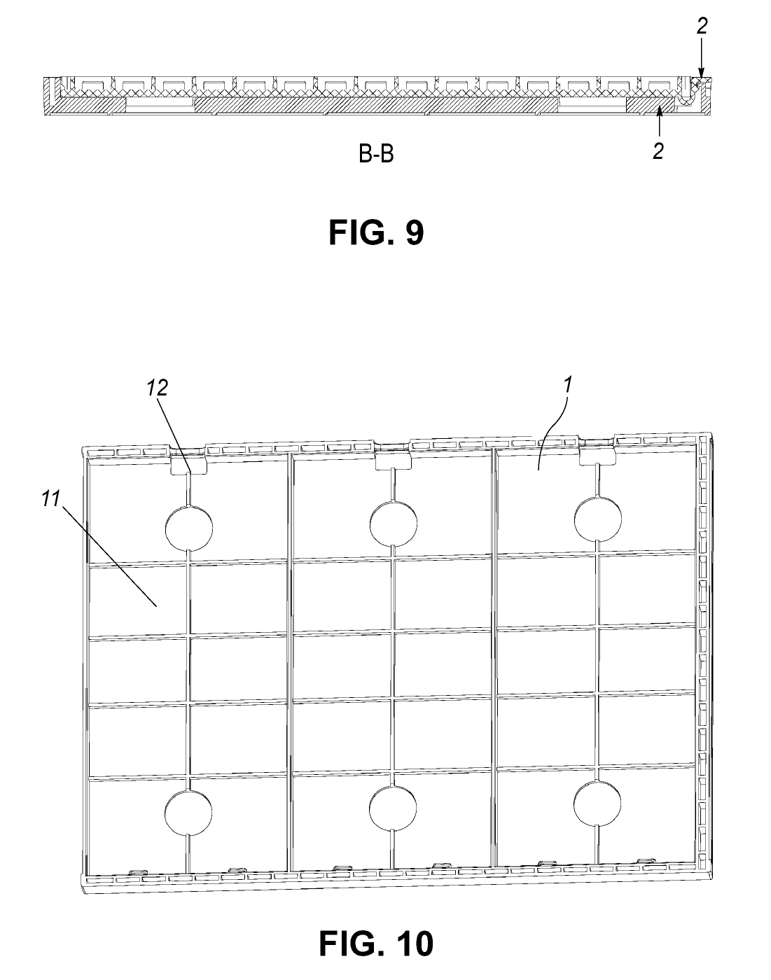

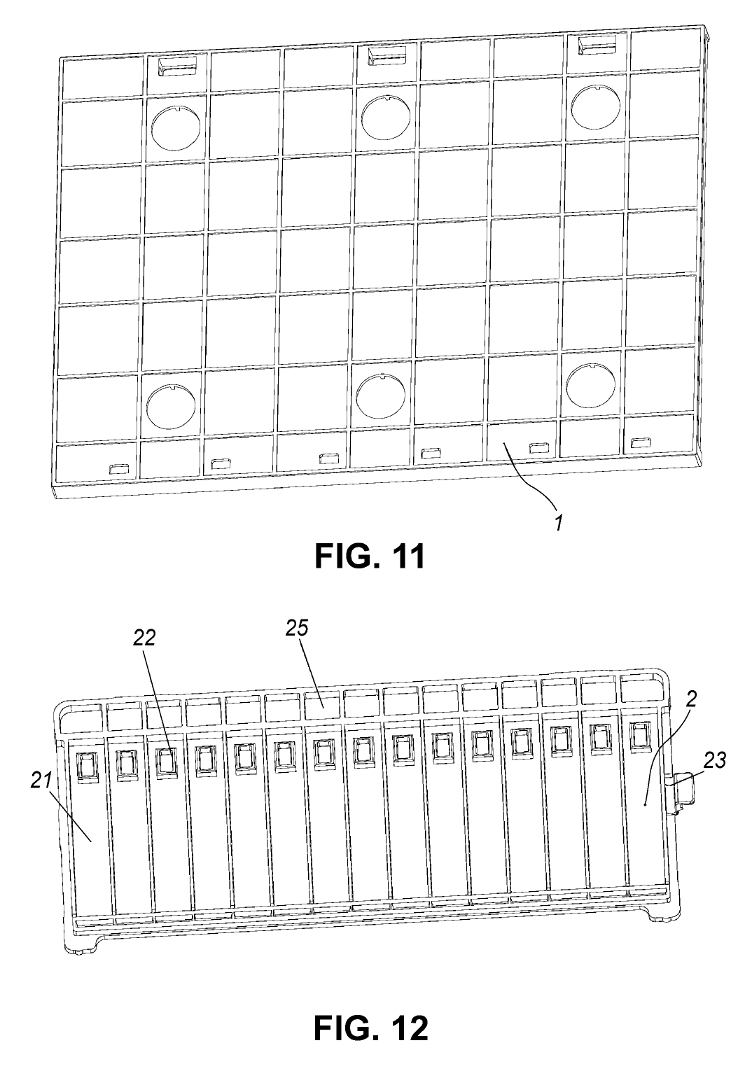

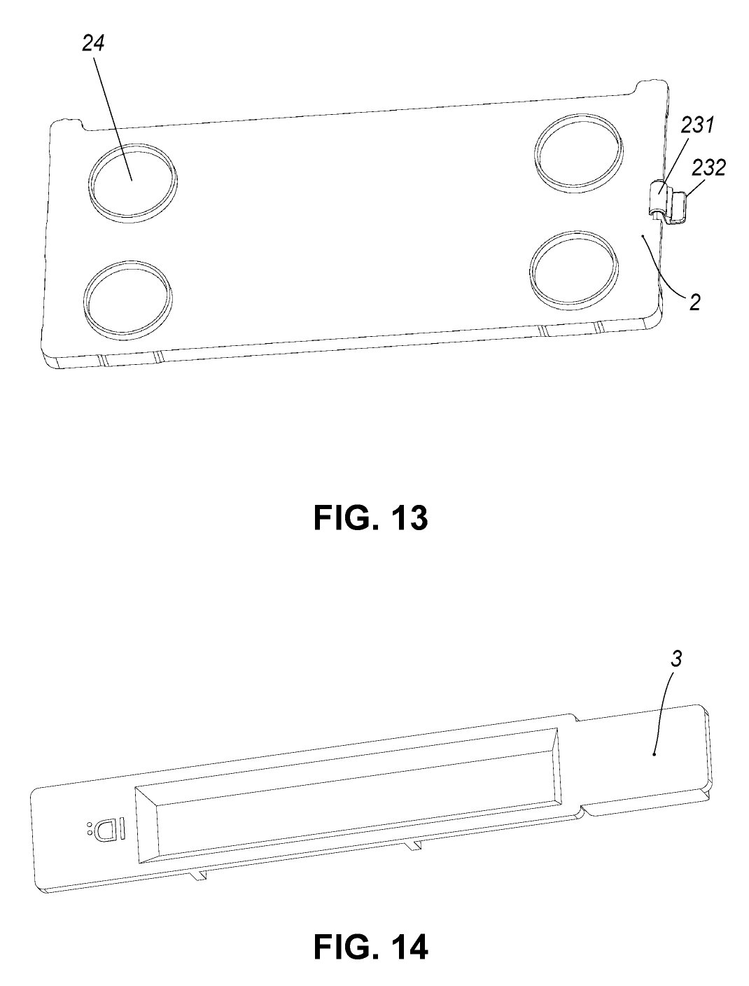

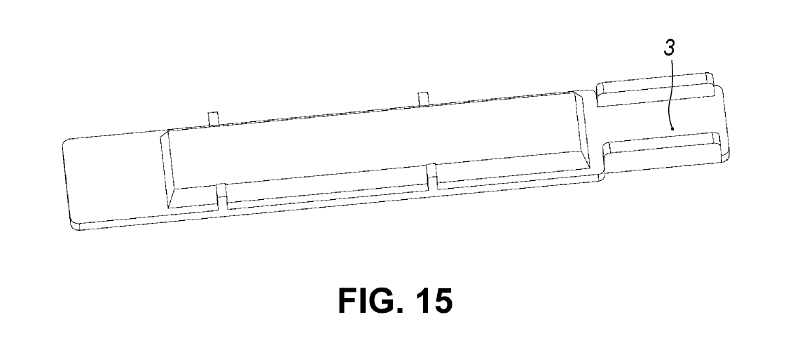

With reference to FIGS. 1-15:

### Embodiment 1

An immunoblotting test board reaction device of the present invention comprises a tray rack 1 and at least one tray 2. A plurality of grooves 21 for receiving test boards are provided in the upper surface of the tray 2. The floor of each groove 21 is equipped with a bayonet 22 for locking the corresponding test board, and the groove 21 of the tray 2 fits snugly to the corresponding test board 3. A snap button 23 for snapping onto the tray rack 1 is provided on one side of the tray 2. Tray back bayonets 24 for placing the tray onto a full-automatic immunoblotting instrument are provided on the bottom surface of the tray 2. The snap button 23 comprises a buckling part 231 and a pressing part 232. One end of the buckling part 231 is fixed to the edge of the tray 2, and the other end is fixedly connected to the pressing part 232. The tray rack 1 comprises tray grooves 11 and snap bayonets 12. The snap bayonets 12 are arranged at the edges of the tray grooves 11 and can be snap-fitted to the buckling parts 231 of the trays 2.

All the grooves 21 of the tray are uniformly arranged in the upper surface of the tray 2 in the same direction, and each groove 21 corresponds to one numbered groove 25.

The numbered grooves 25 are solid or hollow. The numbered grooves may be rectangular, square, semicircular, elliptical, fan-shaped, trapezoidal, rhombic, triangular or in other appropriate shape, and may be numbered in the form of Arabic numerals, lower-case Chinese numerals, Roman numerals or English letters, or in other appropriate form.

Each groove 21 is solid or hollow. At its any position, the groove 21 may have at least one square, rectangular, elliptical, circular, rhombic, triangular or trapezoidal hole or at least one hole in other appropriate shape. The quantity of the holes may be 1, 2, 3 or other appropriate number.

The bayonets 22 may be rectangular, square, semicircle, elliptical, fan-shaped, trapezoidal, rhombic, triangular or in other appropriate shape.

The buckling part 231 is of groove-type structure which may be U-shaped, V-shaped or in other appropriate shape. The pressing part may be rectangular, square, circular, semicircular, elliptical, fan-shaped, trapezoidal, rhombic, triangular or in other appropriate shape, and the snap bayonet 12 may be matched with the pressing part in shape.

At its any position, the bottom surface of the tray 2 may be provided with at least one tray back bayonet 24 for support. The tray back bayonet 24 may be circular, rectangular, square, semicircular, elliptical, fan-shaped, trapezoidal, rhombic, triangular or in other appropriate shape.

Each tray groove 11 of the tray rack 1 is solid or hollow. At its any position, the tray groove 11 of the tray rack 1 may have at least one square, rectangular, elliptical, circular, rhombic, triangular or trapezoidal hole or at least one hole in other appropriate shape.

The trays 2 and the tray rack 1 may be made of ABS, PC, PA, POM, PPE, PETP, PBTP, PPS or other appropriate engineering plastics, or the above described plastics into which 20-30% of or other appropriate ratio of glass fiber material is added.

### Embodiment 2

In this embodiment, an immunoblotting assay is carried out by using the device of the embodiment 1. A test board handle in Inhalant-Food Allergen Specific IgE Antibodies Test Kit 2 (Immunoblotting) is marked with specimen information. The handle on the end of the test board is held by hand. Directing at the test board bayonet of the reaction tray, the test board is then lightly installed into the tray. 1-100 test boards can be placed on each tray at the same time.

Washing solution is added into the test board installed on the tray to fully wet a membrane strip for 3 minutes. After the membrane strip is wet, the numbered groove of the reaction tray is held by one hand and the washing liquid in the test board is poured away and then the test board is spin-dried.

300 microliters of corresponding serum specimen is added into each test board. The tray is placed onto a shaker. After incubation for 45 minutes at room temperature (20-25° C.), the reaction tray is held with one hand and the liquid in the test board is then carefully poured away along the groove. After that, the membrane surface of the test board is repeatedly rinsed 5 times with washing solution in a washing bottle, and then the test board is spin-dried.

300 microliters of second antibody working solution is added into each test board. The tray is placed onto the shaker. After incubation for 45 minutes at room temperature (20-25° C.), the numbered groove of the tray is held with one hand and the liquid in the test board is then carefully poured away along the groove. After that, the membrane surface of the test board is repeatedly rinsed 5 times with washing solution in the washing bottle, and then the test board is spin-dried.

300 microliters of enzyme conjugate is added into each test board. The tray is placed onto the shaker. After incubation for 45 minutes at room temperature (20-25° C.), the numbered groove of the tray is held with one hand and the liquid in the test board is then carefully poured away along the groove. After that, the membrane surface of the test board is repeatedly rinsed 5 times with washing solution in the washing bottle, and then the test board is spin-dried.

300 microliters of substrate solution is added into each test board. The tray is placed onto the shaker. After incubation for 45 minutes at room temperature (20-25° C.), the tray is held with one hand and the solution in the test board is then carefully poured away along the groove. After that, the test board is thoroughly rinsed with running water.

The membrane surfaces of all the test boards and the reaction grooves on the reaction trays are dried with an electric blower. After that, the reaction trays are placed onto the tray rack. The tray rack is then reversely placed onto a scanner, and test results are interpreted via accompanying software.

The contents described in the embodiments of the specification are merely used for particularizing the implementation forms of the present invention. The protection scope of the present invention should not be regarded as being merely limited to the specific forms stated in the embodiments, and the protection scope of the invention also includes equivalent technical means conceivable by those skilled in the art according to the conception of the present invention.

What is claimed is:

1. An immunoblotting test board reaction device, comprising
a tray rack and at least one tray,
wherein, a plurality of grooves for receiving test boards are provided on an upper surface of the tray, a floor of each groove equipped with a bayonet for locking the corresponding test board and the groove of the tray fitting snugly to the corresponding test board;
a snap button for snapping onto the tray rack is provided on one side of the tray, wherein, the snap button comprises a buckling part and a pressing part, one end of the buckling part is fixed to an edge of the tray, and the other end is fixedly connected to the pressing part;
tray back bayonets for placing the tray onto a full-automatic immunoblotting instrument are provided on a bottom surface of the tray; and
the tray rack comprises tray grooves and snap bayonets which are arranged at edges of the tray grooves and can be snap-fitted to the buckling parts of the trays.

2. The immunoblotting test board reaction device according to claim 1, wherein all of the grooves of the tray are uniformly arranged on the upper surface of the tray in the same direction, and each groove corresponds to a numbered groove.

3. The immunoblotting test board reaction device according to claim 2, wherein the numbered grooves are rectangular, square, semicircular, elliptical, fan-shaped, trapezoidal, rhombic or triangular, and are numbered in the form of convex or concave Arabic numerals, lower-case Chinese numerals, Roman numerals or English letters.

4. The immunoblotting test board reaction device according to claim 2, wherein the groove of the tray is provided with at least one square, rectangular, elliptical, circular, rhombic, triangular or trapezoidal hole.

5. The immunoblotting test board reaction device according to claim 1, wherein the bayonets are rectangular, square, semicircular, elliptical, sector-shaped, trapezoidal, rhombic or triangular.

6. The immunoblotting test board reaction device according to claim 1, wherein the buckling parts are of U-shaped or V-shaped groove-type structures, the pressing parts are rectangular, square, circular, semicircular, elliptical, sector-shaped, trapezoidal, rhombic or triangular, and the snap bayonets may be matched with the pressing parts in shape.

7. The immunoblotting test board reaction device according to claim 1, wherein the tray back bayonets are circular, rectangular, square, semicircular, elliptical, sector-shaped, trapezoidal, rhombic or triangular.

8. The immunoblotting test board reaction device according to claim 1, wherein the tray groove of the tray rack is provided with at least one square, rectangular, elliptical, circular, rhombic, triangular or trapezoidal hole.
